(19) United States
(12) Patent Application Publication
(10) Pub. No.: US 2005/0112640 A1
(43) Pub. Date: May 26, 2005

(54) PTD-modified proteins

(75) Inventors: Beverly L. Davidson, North Liberty, IA (US); Qinwen Mao, Iowa City, IA (US); Haibin Xia, Iowa City, IA (US)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: University of Iowa Research Foundation, a Iowa corporation

(21) Appl. No.: 10/952,486

(22) Filed: Sep. 28, 2004

Related U.S. Application Data
(62) Division of application No. 09/757,824, filed on Jan. 9, 2001, now abandoned.

(51) Int. Cl. C12Q 1/68; C07H 21/04; C12N 9/36

(52) U.S. Cl. 435/6; 435/69.1; 435/206; 435/320.1; 435/325; 536/23.2

(57) ABSTRACT

The present invention provides polynucleotides and expression vectors containing a sequence encoding a soluble lysosomal enzyme and a sequence encoding Tat protein transduction domain (PTD), and the corresponding polypeptides. The present demonstrates the utility of these protein fusions in altering the bioavailability of proteins for use in treating genetic diseases or acquired diseases. The invention further provides cell expression systems, and methods of treating a genetic disease or cancer in a mammal using the polynucleotides, polypeptides, or expression system of the present invention.

Portions of the present invention were made with support of the United States Government via a grant from the National Institutes of Health under grant Nos. HD3353 1 and NS34568. The U.S. Government therefore may have certain rights in the invention.

### BACKGROUND OF THE INVENTION

Gene transfer is now widely recognized as a powerful tool for analysis of biological events and disease processes at both the cellular and molecular level. More recently, the application of gene therapy for the treatment of human diseases, either inherited (e.g., ADA deficiency) or acquired (e.g., cancer or infectious disease), has received considerable attention. With the advent of improved gene transfer techniques and the identification of an ever expanding library of “defective gene”-related diseases, gene therapy has rapidly evolved from a treatment theory to a practical reality.

Traditionally, gene therapy has been defined as a procedure in which an exogenous gene is introduced into the cells of a patient in order to correct an inborn genetic error. Although more than 4500 human diseases are currently classified as genetic, specific mutations in the human genome have been identified for relatively few of these diseases. Until recently, these rare genetic diseases represented the exclusive targets of gene therapy efforts. Accordingly, most of the NIH approved gene therapy protocols to date have been directed toward the introduction of a functional copy of a defective gene into the somatic cells of an individual having a known inborn genetic error. Only recently, have researchers and clinicians begun to appreciate that most human cancers, certain forms of cardiovascular disease, and many degenerative diseases also have important genetic components, and for the purposes of designing novel gene therapies, should be considered “genetic disorders.” Therefore, gene therapy has more recently been broadly defined as the correction of a disease phenotype through the introduction of new genetic information into the affected organism.

Two basic approaches to gene therapy have evolved: (1) ex vivo gene therapy and (2) in vivo gene therapy. In ex vivo gene therapy, cells are removed from a subject and cultured in vitro. A functional replacement gene is introduced into the cells (transfection) in vitro, the modified cells are expanded in culture, and then reimplanted in the subject. These genetically modified, reimplanted cells are reported to secrete detectable levels of the transfected gene product in situ. The development of improved retroviral gene transfer methods (transduction) has greatly facilitated the transfer into and subsequent expression of genetic material by somatic cells. Accordingly, retrovirus-mediated gene transfer has been used in clinical trials to mark autologous cells and as a way of treating genetic disease.

In in vivo gene therapy, target cells are not removed from the subject. Rather, the transferred gene is introduced into cells of the recipient organism in situ that is, within the recipient. In vivo gene therapy has been examined in several animal models. Several recent publications have reported the feasibility of direct gene transfer in situ into organs and tissues such as muscle, hematopoietic stem cells, the arterial wall, the nervous system, and lung. Direct injection of DNA into skeletal muscle, heart muscle and injection of DNA-lipid complexes into the vasculature also has been reported to yield a detectable expression level of the inserted gene product(s) in vivo.

Treatment of inherited genetic diseases of the brain remains an intractable problem. An example of such are the lysosomal storage diseases. Collectively, the incidence of lysosomal storage diseases (LSD) is 1 in 12,000 births world wide, and in 58% of cases, there is significant central nervous system (CNS) involvement (Meikle et al., JAMA 281:249-254, 1999). Proteins deficient in these disorders, when delivered intraveneously, do not cross the blood-brain barrier, or, when delivered directly to the brain, are not widely distributed. Injection of viral vectors expressing recombinant lysosomal proteins, a proportion of which is secreted, can result in significant spread of enzyme in murine cerebrum. However, methods to improve the distribution of enzyme following intraventricular injection of recombinant protein, or from transduced cells, are required for approaching therapies in the significantly larger brains of humans. Similar to lysosomal storage diseases, approaching global therapy for degenerative diseases due to polyglutamine repeat expansion or mutations in channels remains a significant problem. Thus, methods to improve the distribution of secreted proteins following transduction of tissues in vivo is required.

### SUMMARY OF THE INVENTION

The present invention provides polynucleotides (DNA or RNA), vectors and polynucleotides encoding a lysosomal enzyme, a secreted protein, a nuclear protein, or a cytoplasmic protein operably linked to a nucleic acid sequence encoding a protein transduction domain (PTD). As used herein, the term “secreted protein” includes any secreted protein, whether naturally secreted or modified to contain a signal sequence so that it can be secreted. Proteins not normally secreted may be modified to contain a secretory signal so that the Tat-protein fusion is secreted out of the cell, where they may then be broadly distributed and contact cellular or intracellular receptors, such as hormone receptors. For example, the secreted protein could be β-glucuruonidase, pepstatin insensitive protease, palmitoyl protein thioesterase. When expressed from the vector the target protein of interest is synthesized in cells, secreted, distributed, and taken up by other cells without a cognate receptor. Soluble lysosomal enzymes are secreted upon overexpression, and can be distributed in vivo when modified to contain the Tat-motif or a similar PTD motif. The PTD can be Tat PTD, and in particular, can be Tat₄₇₋₅₇. The Tat-PTD fusion protein could also be a cytoplasmic protein (such as a cytotoxic agent), a nuclear protein (such as a transcription factor), a growth factor (such as GDNF, BDNF, NGF, or NT3). For example, a nuclear protein could be engineered to be secreted, be taken up by a neighboring cell, and then target the nucleus of the uptaking cell. Alternatively, Tat-PTD could be fused to proteins with anti-neoplastic activity, such as inhibitors of neovascularization, cell migration, or cell proliferation. The fusion proteins may be produced using conventional recombinant DNA technology.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Additionally, multiple copies of the nucleic acid encoding enzymes may be linked together in the expression vector. Such multiple nucleic acids may be separated by linkers. The vector may be an adenoviral vector, an adeno-associated virus (AAV) vector, a retrovirus, or a lentivirus vector based on human immunodeficiency virus or feline immunodeficiency virus. Examples of such AAVs are found in Davidson et al., PNAS (2000) 97:3428-3432. The AAV and lentiviruses could confer lasting expression while the adenovirus would provide transient expression.

The present invention also provides a mammalian cell containing the expression vector described above. The cell may be human, and may be from spleen, kidney, lung, heart, liver or brain. The cell type may be a stem or progenitor cell population.

The present invention provides a method of treating a genetic disease or cancer in a mammal by administering a polynucleotide, polypeptide, expression vector, or cell described above. The genetic disease or cancer may be a lysosomal storage disease (LSD) such as infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease. Alternatively, the genetic disease may be a neurodegenerative disease, such as Huntington's disease, ALS, hereditary spastic hemiplegia, primary lateral sclerosis, spinal muscular atrophy, Kennedy's disease, Alzheimer's disease, a polyglutamine repeat disease, or focal exposure such as Parkinson's disease.

In general, the invention relates to polynucleotides, polypeptides, vectors, and genetically engineered cells (modified ex vivo or in vivo), and the use of them. In particular, the invention relates to a method for gene or protein therapy that is capable of both localized and systemic delivery of a therapeutically effective dose of the therapeutic agent.

According to one aspect of the invention, a cell expression system for expressing a therapeutic agent in a mammalian recipient is provided. The expression system (also referred to herein as a “genetically modified cell”) comprises a cell and an expression vector for expressing the therapeutic agent. Expression vectors of the instant invention include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a recombinant adenoviral, adeno-associated virus, or lentivirus or retrovirus vector.

The expression vector further includes a promoter for controlling transcription of the heterologous gene. The promoter may be an inducible promoter (described below). The expression system is suitable for administration to the mammalian recipient. The expression system may comprises a plurality of non-immortalized genetically modified cells, each cell containing at least one recombinant gene encoding at least one therapeutic agent.

The cell expression system can be formed ex vivo or in vivo. To form the expression system ex vivo, one or more isolated cells are transduced with a virus or transfected with a nucleic acid or plasmid in vitro. The transduced or transfected cells are thereafter expanded in culture and thereafter administered to the mammalian recipient for delivery of the therapeutic agent in situ. The genetically modified cell may be an autologous cell, i.e., the cell is isolated from the mammalian recipient. The genetically modified cell(s) are administered to the recipient by, for example, implanting the cell(s) or a graft (or capsule) including a plurality of the cells into a cell-compatible site of the recipient.

According to yet another aspect of the invention, a method for treating a mammalian recipient in vivo is provided. The method includes introducing an expression vector for expressing a heterologous gene product into a cell of the patient in situ. To form the expression system in vivo, an expression vector for expressing the therapeutic agent is introduced in vivo into target location of the mammalian recipient by, for example, intraperitoneal injection or injection directly into the brain.

According to yet another aspect of the invention, a method for treating a mammalian recipient in vivo is provided. The method includes introducing the recombinant PTD-fusion protein into the tissues of the patient in vivo. The therapeutic agent is introduced in vivo into target location of the mammalian recipient by, for example, a pump to provide continuous delivery into brain ventricles.

The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

The mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid or protein components.

According to one embodiment, the mammalian recipient has a genetic disease and the exogenous genetic material comprises a heterologous gene encoding a therapeutic agent for treating the disease. In yet another embodiment, the mammalian recipient has an acquired pathology and the exogenous genetic material comprises a heterologous gene encoding a therapeutic agent for treating the pathology. According to another embodiment, the patient has a cancer and the exogenous genetic material comprises a heterologous gene encoding an anti-neoplastic agent. In yet another embodiment the patient has an undesired medical condition and the exogenous genetic material comprises a heterologous gene encoding a therapeutic agent for treating the condition.

According to yet another embodiment, a pharmaceutical composition is disclosed. The pharmaceutical composition comprises a plurality of the above-described genetically modified cells or polypeptides and a pharmaceutically acceptable carrier. The pharmaceutical composition may be for treating a condition amenable to gene replacement therapy and the exogenous genetic material comprises a heterologous gene encoding a therapeutic agent for treating the condition. The pharmaceutical composition may contain an amount of genetically modified cells or polypeptides sufficient to deliver a therapeutically effective dose of the therapeutic agent to the patient. Exemplary conditions amenable to gene replacement therapy are described below.

According to another aspect of the invention, a method for forming the above-described pharmaceutical composition is provided. The method includes introducing an expression vector for expressing a heterologous gene product into a cell to form a genetically modified cell and placing the genetically modified cell in a pharmaceutically acceptable carrier.

According to still another aspect of the invention, a cell graft is disclosed. The graft comprises a plurality of genetically modified cells attached to a support, which is suitable for implantation into the mammalian recipient. The support may be formed of a natural or synthetic material.

According to still another aspect of the invention, an encapsulated cell expression system is disclosed. The encapsulated expression system comprises a plurality of genetically modified cells contained within a capsule, which is suitable for implantation into the mammalian recipient. The capsule may be formed of a natural or synthetic material. The capsule containing the plurality of genetically modified cells may be implanted in the peritoneal cavity, the brain or ventricles in the brain, or into the specific disease site.

According to still another aspect of the invention, a protein delivery method is disclosed. The protein is purified from genetically modified cells and then placed into the mammalian recipient. The purified protein is placed into the brain, into the peritoneum, or into the specific disease site.

These and other aspects of the invention as well as various advantages and utilities will be more apparent with reference to the detailed description of the invention and to the accompanying Figures.

As used herein, the term “lysosomal enzyme,” a “secreted protein,” a “nuclear protein,” a “cytoplasmic protein,” or a “Tat protein transduction domain” include variants or biologically active or inactive fragments of these polypeptides. A “variant” of one of the polypeptides is a polypeptide that is not completely identical to a native protein. Such variant protein can be obtained by altering the amino acid sequence by insertion, deletion or substitution of one or more amino acid. The amino acid sequence of the protein is modified, for example by substitution, to create a polypeptide having substantially the same or improved qualities as compared to the native polypeptide. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spacial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains. Stryer, L. Biochemistry (2d edition) W. H. Freeman and Co. San Francisco (1981), p. 14-15; Lehninger, A. Biochemistry (2d ed., 1975), p. 73-75.

The amino acid changes are achieved by changing the codons of the corresponding nucleic acid sequence. It is known that such polypeptides can be obtained based on substituting certain amino acids for other amino acids in the polypeptide structure in order to modify or improve biological activity. For example, through substitution of alternative amino acids, small conformational changes may be conferred upon a polypeptide which result in increased. Alternatively, amino acid substitutions in certain polypeptides may be used to provide residues, which may then be linked to other molecules to provide peptide-molecule conjugates which, retain sufficient properties of the starting polypeptide to be useful for other purposes.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated in intended for use in immunological embodiments. The greatest local average hydrophilicity of a “protein”, as governed by the hydrophilicity of its adjacent amino acids, correlates with its immunogenicity. U.S. Pat. No. 4,554,101. Accordingly, it is noted that substitutions can be made based on the hydrophilicity assigned to each amino acid.

In using either the hydrophilicity index or hydropathic index, which assigns values to each amino acid, it is preferred to conduct substitutions of amino acids where these values are ±2, with ±1 being particularly preferred, and those with in ±0.5 being the most preferred substitutions.

The variant protein has at least 50%, at least about 80%, or even at least about 90% but less than 100%, contiguous amino acid sequence homology or identity to the amino acid sequence of a corresponding native protein.

The amino acid sequence of the variant polypeptide corresponds essentially to the native polypeptide's amino acid sequence. As used herein “correspond essentially to” refers to a polypeptide sequence that will elicit a biological response substantially the same as the response generated by the native protein. Such a response may be at least 60% of the level generated by the native protein, and may even be at least 80% of the level generated by native protein.

A variant of the invention may include amino acid residues not present in the corresponding native protein or deletions relative to the corresponding native protein. A variant may also be a truncated “fragment” as compared to the corresponding native protein, i.e., only a portion of a full-length protein. Protein variants also include peptides having at least one D-amino acid.

The variant protein of the present invention may be expressed from an isolated DNA sequence encoding the variant protein. “Recombinant” is defined as a peptide or nucleic acid produced by the processes of genetic engineering. It should be noted that it is well-known in the art that, due to the redundancy in the genetic code, individual nucleotides can be readily exchanged in a codon, and still result in an identical amino acid sequence. The terms “protein,” “peptide” and “polypeptide” are used interchangeably herein.

### BRIEF DESCRIPTION OF THE DRAWINGS

FIG. 1. β-glucuronidase-Tat expression vectors. (a) Cartoon depicting the orientation of the Tat motifs at the carboxy termini of β-glucuronidase. The β-glucuronidase sequences were cloned into the E1 region of Ad shuttle plasmids, and the shuttles recombined with Ad backbones expressing GFP in the E3 region. The resultant viruses expressed β-glucuronidase, β-glucuronidase-Tat₄₇₋₅₇ or β-glucuronidase-Tat₅₇₋₄₇ in E1 and GFP in E3. Both trangenes are driven off the RSV promoter. (b-d), β-glucuronidase activity after incubation of A549 cells with the recombinant proteins β-glucuronidase, β-glucuronidase-Tat₄₇₋₅₇ or β-glucuronidase-Tat₅₇₋₄₇, respectively. Using the assay conditions described in the Examples below the background levels of β-glucuronidase staining is very low (inset, panel b). The uptake of both native and tat-modified β-glucuronidase (inset, panel d) was notably punctate. (e-g), β-glucuronidase activity after incubation of A549 cells with the recombinant proteins β-glucuronidase, β-glucuronidase-Tat₄₇₋₅₇ or β-glucuronidase-Tat₅₇₋₄₇ in the presence of D-mannose-6-phosphate. Bars=50 μm.

FIG. 2. eGFP and β-glucuronidase activity in sections of murine liver after i.v. injection of vectors expressing native or Tat-modified β-glucuronidase. (a-c), photomicrographs showing representative levels of GFP expression in murine liver following injection of Adβgluc, AdβglucTat₄₇₋₅₇ or AdβglucTat₅₇₋₄₇, respectively. (d-f), sections from mice transduced with Adβgluc, AdβglucTat₄₇₋₅₇ or AdβglucTat₅₇₋₄₇, respectively, stained in situ for β-glucuronidase activity. Bar=200 μm.

FIG. 3. β-glucuronidase activity in non-hepatic tissues after i.v. injection of mice with vectors expressing native or Tat-modified β-glucuronidase. β-glucuronidase activity was detected in situ ten days after i.v. injection of Adβgluc (a,c,e,g,i) or Adβgluc-Tat₄₇₋₅₇ (b,d,f,h,j). Representative sections from spleen (a,b), kidney (c,d) lung (e,f), heart (g,h) and brain (i,j) are shown. Scale bar is 400 μm. (k), enzyme activity levels in tissue lysates.

FIG. 4. GFP and β-glucuruonidase distribution and activity in brain. Mice were injected with Adβgluc (a,c), Adβgluc-Tat₄₇₋₅₇ (b,d) or Adβgluc-Tat₅₇₋₄₇ into straita, and GFP and β-glucuronidase activity evaluated ten days later on full corona sections (a-e) or tissue lysates (f). Equivalent i.u. (and particles) were injected. Sections photomicrographed in c and d are within 60 μm from those shown in a and b, respectively. (e), the volume of brain positive for GFP and β-glucuronidase quantified using NIH Image. (f), enzyme activities for the contralateral (CL) and injected hemispheres (IL) were determined as described in Methods, and expressed as CL/(CL+IL)×100.

FIG. 5. Expression of β-glucuronidase or β-glucuronidase-Tat₄₇₋₅₇ from transduced ependyma. Mice were injected with Adβgluc (a,d), Adβgluc-Tat₄₇₋₅₇ (b,e) or Adβgluc-Tat₅₇₋₄₇ (c,f) and brains harvested ten days later for evaluation of GFP (a-c) or β-glucuronidase (d-f) expression. Sections photomicrographed in a-c are within 60 μm from those shown in d-f. The volume of brain (both hemispheres) positive for β-glucuronidase activity was determined using NIH image.

FIG. 6. Expression of β-glucuronidase and β-glucurondiase-Tat in the brainstem. Mice were injected with Adβgluc or Adβgluc-Tat₄₇₋₅₇ and animals sacrificed ten days later for evaluation of GFP or β-glucuronidase expression.

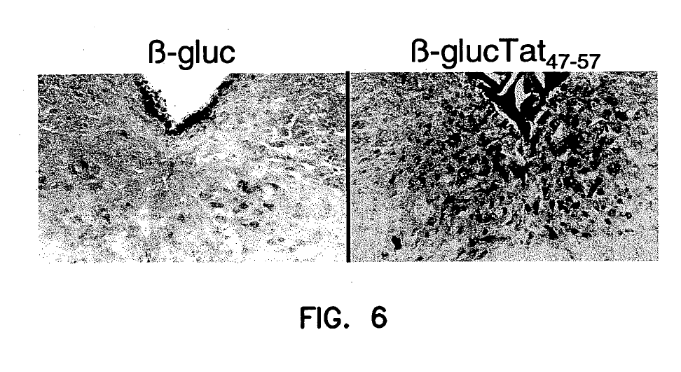

### DETAILED DESCRIPTION OF THE INVENTION

Collectively, the prevalence of lysosomal storage diseases is strikingly high. As an example, a 16 year retrospective study in Australia revealed a prevalence between 1 in 6,700 to 1 in 7700 live births (Meikle, et al., (1999) JAMA 281(3):249-254). In 58% of cases, there is significant CNS involvement. Early work in rodent models of the lysosomal storage diseases has shown tremendous promise in addressing the systemic manifestations of these disorders, either by enzyme replacement or bone marrow transplant to adult recipients. However these therapies did not ameliorate or substantially delay progressive neurodegeneration. In the β-glucuronidase deficient mouse, inhibition of cognitive decline required that treatment be initiated in the neonatal period systemically prior to blood-brain barrier (BBB) closure (O'Connor, et al., (1998) J. Clin.Invest. 101:1394-1400), or directly to brain (Frisella, et al., (2001) Mol. Ther. (In Press)).

Recent work showed that the 11 amino acid motif from HIV Tat known as the protein transduction domain (PTD) improved the biodistribution of recombinant reporter proteins following systemic delivery (Fawell, et al., (1994). Proc.Natl.Acad.Sci.U.S.A. 91:664-668), (Schwarze, et al., (1999) Science 285:1569-1572). When partially denatured, the protein was capable of crossing the blood brain barrier of adult mice (Schwarze, et al., (1999) Science 285:1569-1572). These findings suggest that gene therapy with vectors engineered to express Tat-modified recombinant lysosomal proteins from systemic sources in vivo could be used to improve their biodistribution.

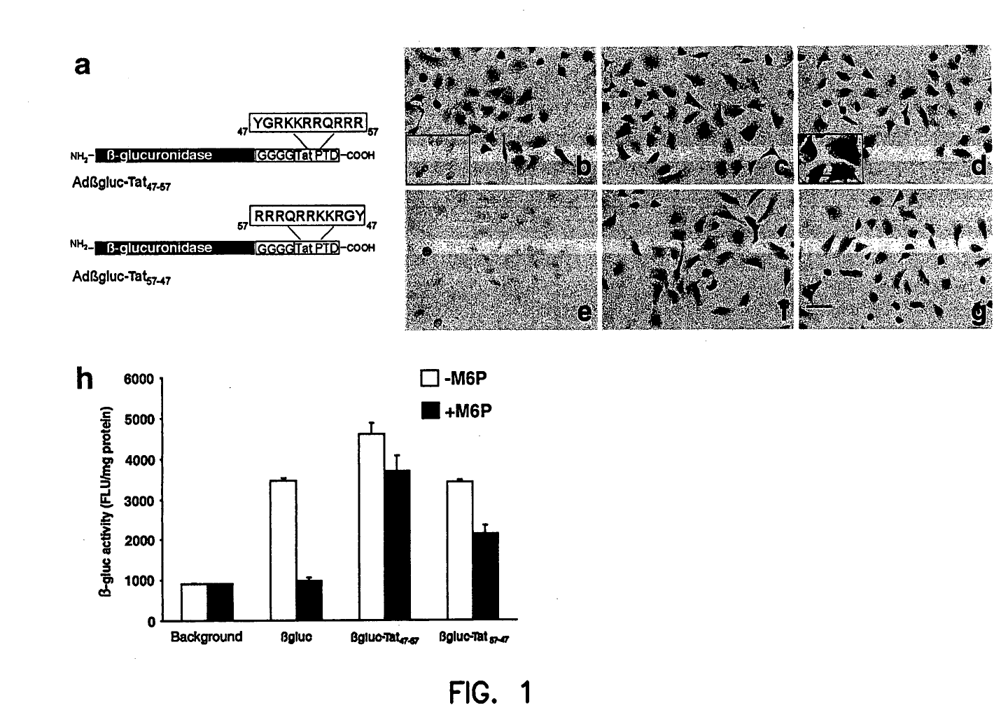

To test this, fusion proteins of human β-glucuronidase and the 11 amino acid PTD from HIV Tat were engineered in recombinant adenovirus expression vectors (FIG. 1a). As peptides representative of the PTDs from Drosophila antenapedea can translocate across cell membranes in either orientation (Derossi, et al., (1996) J.Biol.Chem. 271(30):18188-18193) fusion proteins with the HIV Tat peptide in the 47-57 and 57-47 orientation were generated. We first examined the properties of the modified β-glucuronidase for mannose-6 phosphate (M6P) dependent and independent entry into cells. HeLa cells were infected with 20 infectious units (i.u.)/cell of recombinant vectors expressing unmodified β-glucuronidase (Adβgluc), β-glucuronidase-Tat₄₇₋₅₇ (Adβgluc-Tat₄₇₋₅₇) or β-glucuronidase-Tat₅₇₋₄₇ (Adβgluc-Tat₅₇₋₄₇). Three days later, supernatants were collected and β-glucuronidase activity quantified. The Tat modification to the COOH-terminus did not inhibit enzyme activity. Equivalent units of β-glucuronidase, β-glucuronidase-Tat₄₇₋₅₇ or β-glucuronidase-Tat₅₇₋₄₇ were added to the media of A549 cells in the presence or absence of M6P (FIG. 1b-g). While all recombinant proteins entered cells readily (FIG. 1b-d), M6P dramatically inhibited the uptake of native β-glucuronidase (FIG. 1e) relative to β-glucuronidase-Tat₄₇₋₅₇ or β-glucuronidase-Tat₅₇₋₄₇ (FIG. 1f,g) as assayed by an in situ activity stain (Ghodsi, et al., (1998) Hum. Gene Ther. 9:2331-2340). Quantitation of enzyme activity showed that M6P inhibited 100% of uptake of native β-glucuronidase. β-Glucuronidase-Tat₄₇₋₅₇ or β-glucuronidase-Tat₅₇₋₄₇, were inhibited by 24 and 51%, respectively (FIG. 1h). Thus β-glucuronidase modified at the COOH terminus with the PTD of Tat allowed for both M6P dependent and independent entry. Similar results were found when the wild type and Tat-modified β-glucuronidase-containing supernatants were added to cultures of NIH 3T3 cells with or without M6P.

Earlier studies showed that uptake of Tat-modified proteins occurred by adsorptive endocytosis in cell lines and primary cell cultures (Fawell, et al., (1994) Proc.Natl.Acad.Sci. U.S.A. 91:664-668), (Mann, et al., (1991) EMBO J. 10(7):1733-1739). Mann and Frankel also showed that entry of [¹²⁵I] Tat was temperature dependent (Mann, et al., (1991) EMBO J. 10(7):1733-1739). This is distinct from peptides representative of the PTD from antenopedia, which enters cells readily at 4 and 37° C. (Derossi, et al., (1996) J.Biol.Chem. 271(30):18188-18193). Equivalent units of β-glucuronidase, β-glucuronidase-Tat₄₇₋₅₇ or β-glucuronidase-Tat₅₇₋₄₇ were added to cells and uptake at 4 and 37° C. measured and compared. In all cases, enzyme uptake at 4° C. was dramatically inhibited compared to that occurring at 37° C. These data, and the observation that histochemical staining for enzyme activity at time points early after enzyme addition was punctate (FIG. 1d, inset), suggests that Tat-modified β-glucuronidase, like native β-glucuronidase, enters cells in part through endocytic mechanisms.

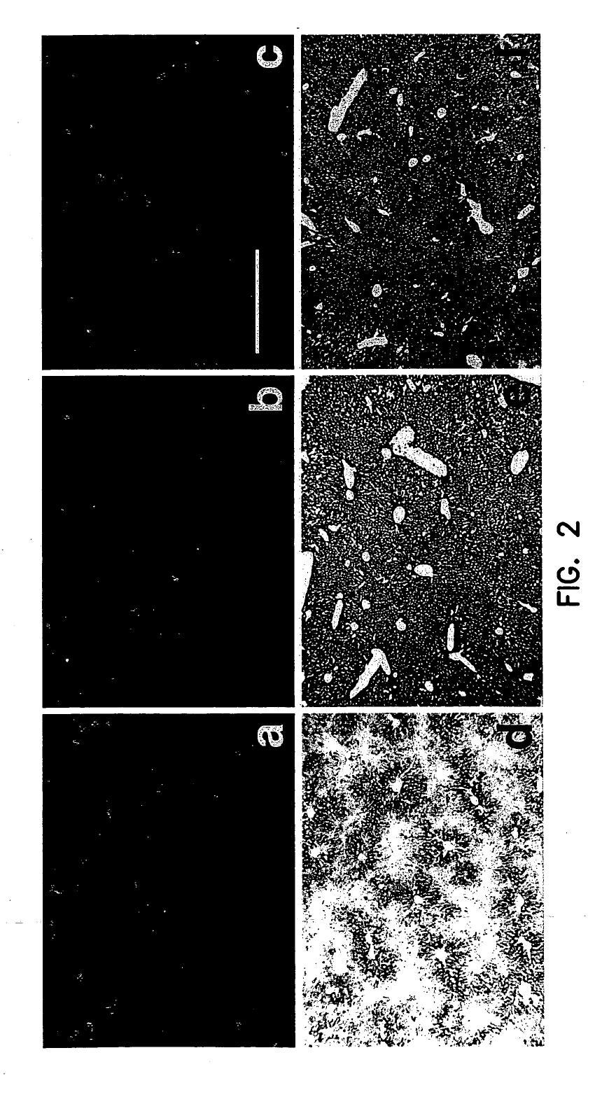

We next investigated Adβgluc, Adβgluc-Tat₄₇₋₅₇ and Adβgluc-Tat₅₇₋₄₇ in vivo. Viruses were injected into mice tail veins, which results in transduction of hepatocytes (Stein, et al., (1999) J. Virol. 73(4):3424-3429). The vectors used in this study also expressed GFP in the E3 region to permit detection of infected cells (GFP positive) relative to β-glucuronidase, β-glucuronidase-Tat₄₇₋₅₇ or β-glucuronidase-Tat₅₇₋₄₇ activity. Sections of liver analyzed 10 days after i.v. vector injection show roughly equivalent levels of GFP expression for all viruses (FIG. 2a-c), but varied distribution of β-glucuronidase activity (FIG. 2d-f). β-Glucuronidase-Tat₄₇₋₅₇ and β-glucuronidase-Tat₅₇₋₄₇ activity were detected throughout the parenchyma of the liver as evidenced by in situ enzyme activity assay (FIG. 2e,f). In contrast, transduction with Adβgluc resulted in focal staining (FIG. 2d).

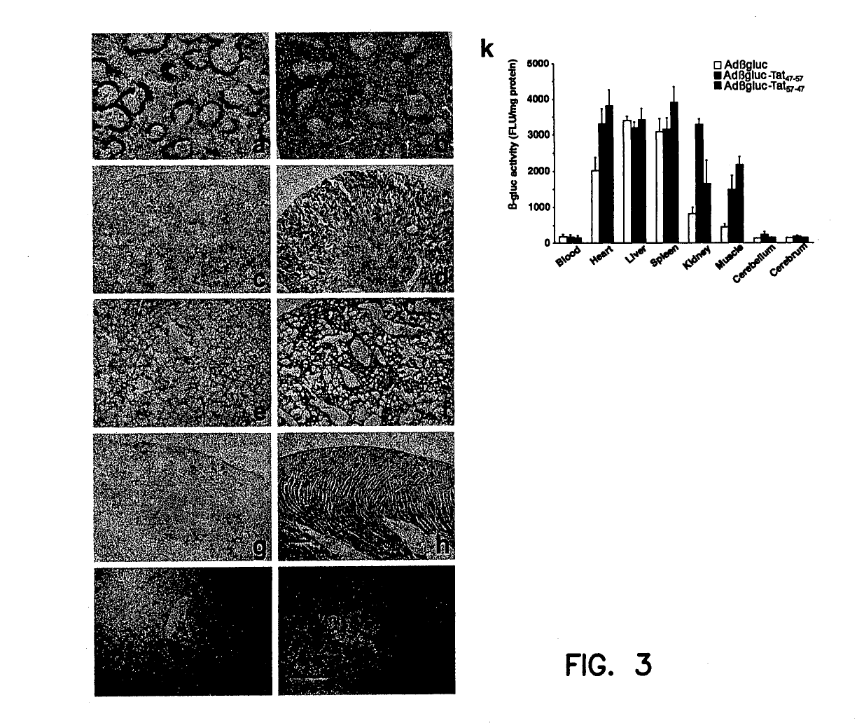

Similar to native β-glucuronidase (Stein, et al., (1999) J. Virol. 73(4):3424-3429), we also noted spread of β-glucuronidase-Tat₄₇₋₅₇ and β-glucuronidase-Tat₅₇₋₄₇ to other tissues (FIG. 3). In some instances the penetration of the enzyme within specific organs or tissues was remarkably distinct from native β-glucuronidase. For example, in the spleen (FIG. 3a,b), extensive β-glucuronidase activity was found in the marginal zone and to a limited extent in the red pulp after transduction with Adgluc. However, β-glucuronidase-Tat₄₇₋₅₇ fully penetrated the red-pulp (FIG. 3b). β-glucuronidase-Tat₅₇₋₄₇ was comparable. Interestingly, β-glucuronidase-Tat₄₇₋₅₇ distribution was similar to sections from mice receiving i.p. injections of partially denatured, purified E. coli β-galactosidase-Tat fusion proteins (Schwarze, et al., (1999) Science 285:1569-1572).

We also noted increased levels of enzyme in kidney (FIG. 3c,d), lung (FIG. 3e,f) heart (FIG. 3g,h), and skeletal muscle for β-glucuronidase-Tat₄₇₋₅₇ and β-glucuronidase-Tat₅₇₋₄₇. Although the distribution of β-glucuronidase activity was widespread in kidney and lung in AdβglucTat₄₇₋₅₇ vs. Adβgluc treated mice, β-glucuronidase activity remained undetectable in both lung lavage fluid and urine.

In contrast to earlier studies with recombinant protein (Schwarze, et al., (1999) Science 285:1569-1572), we noted only a modest increase in enzyme staining in brain, all limited to the choroid plexus (FIG. 3i,j). Quantitative enzyme assay of brain lysates indicated that there were no significant differences between the treatment groups (FIG. 3k). Together the data suggest that the 11 amino acid PTD from Tat may alter the biodistribution of native proteins expressed and secreted in vivo from transduced cells. However the addition of the Tat motif to β-glucuronidase, expressed from systemically transduced tissues of adult mice, does not significantly improve enzyme levels within brain. Possibilities for the discrepancies include differences in the type of protein delivered. Dowdy and colleagues achieved penetration of the blood brain barrier with denatured/partially renatured β-galactosidase. Fawell and colleagues used native β-galactosidase-Tat conjugates in their studies, and did not see penetration of the brain. Both studies delivered approximately 4×10⁻⁹ mole of β-galactosidase by intraperitoneal injection, for an estimated serum concentration of 1 μM. In our studies, the Tat-modified β-glucuronidase reached an approximate serum concentration of 16 nM, and likely remained in native conformation.

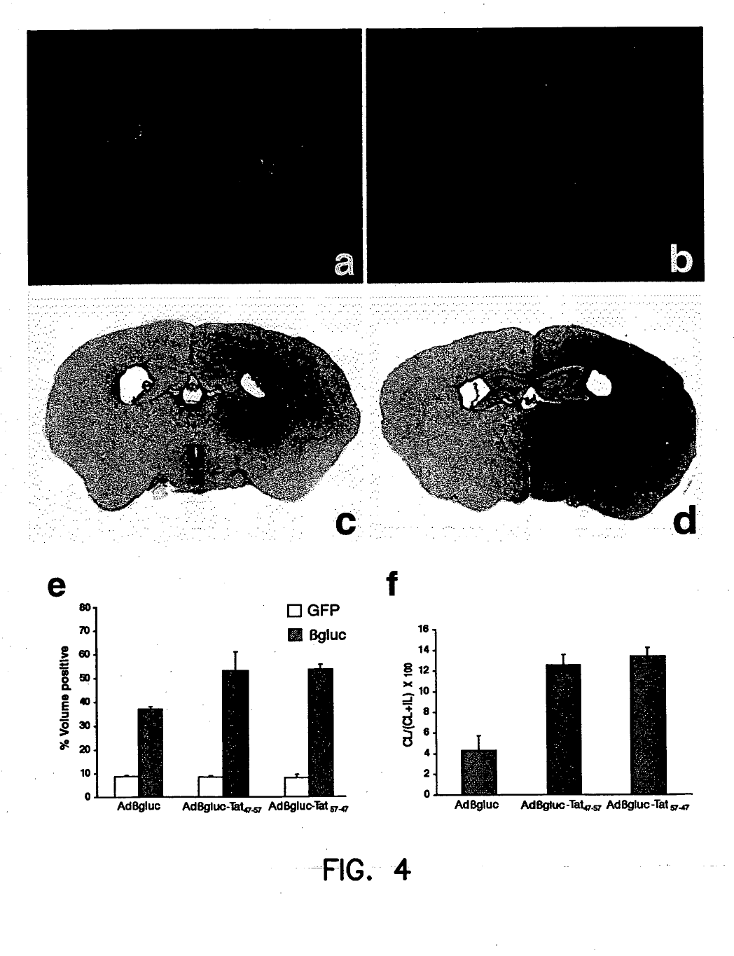

It is not known if the partially-denatured, Tat-modified reporters described by Dowdy and colleagues can pass an intact blood brain barrier in larger animal models. It would also be important to know if the Tat-motifs could impart improved distribution of proteins when administered directly to, or expressed from, cells within the brain. To determine this, vectors expressing β-glucuronidase, β-glucuronidase-Tat₄₇₋₅₇ or β-glucuronidase-Tat₅₇₋₄₇ (2×10⁷ i.u.) were injected into the right hemisphere, and animals sacrificed 10 days later. All vectors yielded nearly equivalent levels of GFP expression (FIG. 4a,b). However, the addition of the Tat motif to β-glucuronidase resulted in significantly greater distribution of enzyme compared to the non-modified protein (FIG. 4.c vs. d). As a consequence, there was a 1.5 - fold increase in the volume of brain positive for β-glucuronidase activity (FIG. 4e), and a notable increase in the levels of β-glucuronidase activity in the contralateral hemisphere (FIG. 4f).

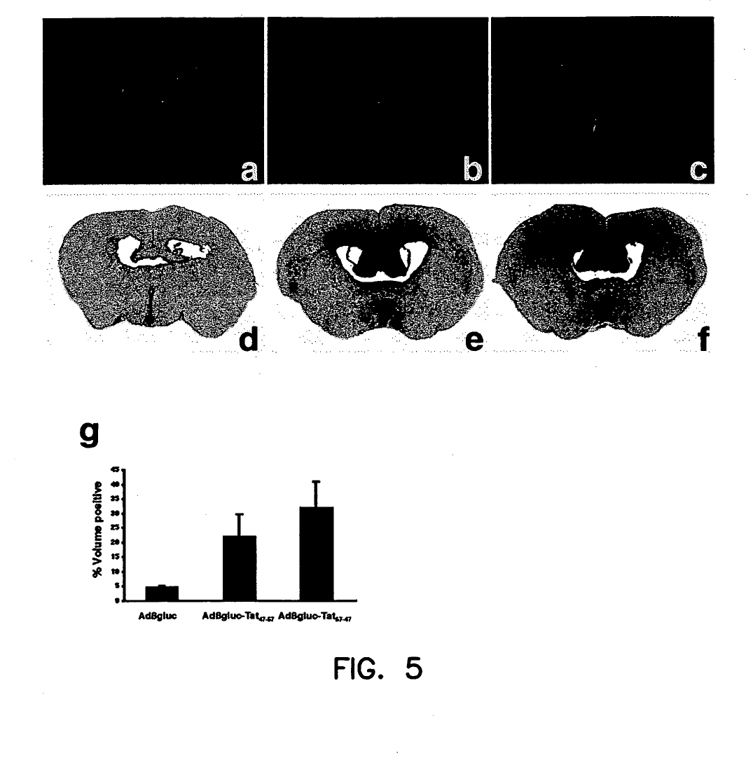

Ventricular administration of secreted proteins for the MPS or other lysosomal storage diseases would be preferred over multiple parenchymal injections if adequate spread of enzyme into the parenchyma can occur. Injection of the recombinant vectors into the lateral ventricles of mice led to significant transduction of ependyma as evidenced by GFP fluorescence (FIG. 5a,c) (Ghodsi, et al., (1999) Exp.Neurol. 160:109-116). As shown previously, β-glucuronidase expression from Adβgluc was obvious in areas immediately adjacent to the ependyma (FIG. 5d). However, the penetration of β-glucuronidase-Tat was remarkably enhanced, resulting in significant increases in the volume of brain positive for active enzyme (FIG. 5g). In animals receiving intraventricular injection of Adβgluc, 5% of the brain was β-glucuronidase positive. In contrast, expression of β-glucuronidase-Tat₄₇₋₅₇ or β-glucuronidase-Tat₅₇₋₄₇ was distributed in 22 and 30% of the brain, respectively. Increased distribution of expressed enzyme after intraventricular injection has important implications for enzyme-based therapy or for gene therapy using vectors with high affinity to the ependymal lining, such as recombinant adenoviruses (Ghodsi, et al., (1999) Exp.Neurol. 160:109-116) and adeno-associated virus type 4 (Davidson, et al., (2000) Proc.Natl.Acad.Sci. U.S.A. 97(7):3428-3432).

Prior to this work, PTDs had been applied as synthetic peptides or used to improve transfer of nuclear and cytoplasmic proteins. We show that the PTD from HIV Tat allowed for significant improvements in the distribution of a lysosomal protein expressed and secreted from cells after viral-mediated gene transfer to liver and brain. When ependyma lining the ventricles were transduced, there was a 5 to 7 fold increase in the volume of brain positive for β-glucuronidase activity. Thus PTDs could also dramatically improve the biodistribution of recombinant enzyme following intraventricular injection. Together, our data represent a significant improvement in the development of gene and protein therapies for inherited genetic diseases affecting the brain.

The present invention provides methods of treating a genetic disease or cancer in a mammal by administering a polynucleotide, polypeptide, expression vector, or cell. For the gene therapy methods, a person having ordinary skill in the art of molecular biology and gene therapy would be able to determine, without undue experimentation, the appropriate dosages and routes of administration of the polynucleotide, polypeptide, or expression vector used in the novel methods of the present invention.

The instant invention provides a cell expression system for expressing exogenous genetic material in a mammalian recipient. The expression system, also referred to as a “genetically modified cell”, comprises a cell and an expression vector for expressing the exogenous genetic material. The genetically modified cells are suitable for administration to a mammalian recipient, where they replace the endogenous cells of the recipient. Thus, the genetically modified cells may be non-immortalized and are non-tumorigenic.

According to one embodiment, the cells are transformed or otherwise genetically modified ex vivo. The cells are isolated from a mammal (for example, a human), transformed (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transformed or otherwise genetically modified in vivo. The cells from the mammalian recipient are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into anti-sense RNA, as well as a “heterologous gene” (i.e., a gene encoding a protein which is not expressed or is expressed at biologically insignificant levels in a naturally-occurring cell of the same type).

In the certain embodiments, the mammalian recipient has a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid (e.g., antisense RNA) and/or protein components.

A number of lysosomal storage diseases are known (for example Neimann-Pick disease, Sly syndrome, Gaucher Disease). Other examples of lysosomal storage diseases are provided in Table 1. Therapeutic agents effective against these diseases are also known, since it is the protein/enzyme known to be deficient in these disorders.

TABLE 1
List of putative target diseases for PTD-based therapies.

| Disease^{a} | Post-natal^{b} | % |
|---|---|---|
| Gaucher | 71 | 13.0 |
| Juvenile Batten | 39 | 7.2 |
| Fabry | 36 | 6.6 |
| MLD | 35 | 6.4 |
| Sanfilippo A | 33 | 6.1 |
| Late Infantile Batten | 27 | 5.0 |
| Hunter | 26 | 4.8 |
| Krabbe | 21 | 3.9 |
| Morquio | 21 | 3.9 |
| Pompe | 21 | 3.9 |
| Niemann-Pick C | 20 | 3.7 |
| Tay-Sachs | 19 | 3.5 |
| Hurler (MPS-I H) | 18 | 3.3 |
| Sanfilippo B | 18 | 3.3 |
| Maroteaux-Lamy | 17 | 3.1 |
| Niemann-Pick A | 16 | 2.9 |
| Cystinosis | 15 | 2.8 |
| Hurler-Scheie (MPS-I H/S) | 10 | 1.8 |
| Sly Syndrome (MPS VII) | 0 | 0 |
| Scheie (MPS-I S) | 10 | 1.8 |
| Infantile Batten | 10 | 1.8 |
| GM1 Gangliosidosis | 10 | 1.8 |
| Mucolipidosis type II/III | 10 | 1.8 |
| Sandhoff | 10 | 1.8 |
| other | 32 | 5.9 |

% LSDs with CNS involvement 58.317757
^{a}Diseases tested in a 16 year retrospective study in Australia (Menke et al, JAMA).
^{b}prevalence from post-natal diagnoses only

As used herein, “acquired pathology” refers to a disease or syndrome manifested by an abnormal physiological, biochemical, cellular, structural, or molecular biological state. Exemplary acquired pathologies, are provided in Table 2. Therapeutic agents effective against these diseases are also given.

TABLE II
Potential Gene Therapies for Motor Neuron Diseases and other
neurodegenerative diseases.

| | | Candidates for | Neuronal or |
|---|---|---|---|
| | Candidates for Gene | Downstream | Progenitor Cell |
| Disease | Replacement² | Effectors³ | Replacement⁴ |
| ALS | No | Yes | Yes |
| Hereditary | Spastin, paraplegin | Yes | Yes |
| spastic | | | |
| hemiplegia | | | |
| Primary lateral | No | Yes | Yes |
| sclerosis⁵ | | | |
| Spinal | Survival motor neuron | Yes | Yes |
| muscular | gene, neuronal | | |
| atrophy | apoptosis inhibiting | | |
| | factor | | |
| Kennedy's | Androgen-receptor | Yes | Yes |
| disease | element | | |
| Alzheimer's | | Yes | Yes |
| disease | | | |
| Polyglutamine | | Yes | Yes |
| Repeat | | | |
| Diseases | | | |

²Based on current literature.
³Based on current literature, includes calbindin, trophic factors, bcl-2, neurofilaments, and pharmacologic agents.
⁴May include cell- or cell- and gene-based therapies.
⁵A sporadic degeneration of corticospinal neurons, 1/100^{th} as common as ALS, with no known genetic links.

Delivery of a therapeutic agent by a genetically modified cell is not limited to delivery to a particular location in the body in which the genetically modified cells would normally reside. Accordingly, the genetically modified cells of the invention are useful for delivering a therapeutic agent, such as a replacement protein, an anti-neoplastic agent, or a neuroprotective agent, to various parts or the appropriate part of the body.

Alternatively, the condition amenable to gene replacement therapy is a prophylactic process, i.e., a process for preventing disease or an undesired medical condition. Thus, the instant invention embraces a cell expression system for delivering a therapeutic agent that has a prophylactic function (i.e., a prophylactic agent) to the mammalian recipient.

In summary, the term “therapeutic agent” includes, but is not limited to, the agents listed in the Tables above, as well as their functional equivalents. As used herein, the term “functional equivalent” refers to a molecule (e.g., a peptide or protein) that has the same or an improved beneficial effect on the mammalian recipient as the therapeutic agent of which is it deemed a functional equivalent. As will be appreciated by one of ordinary skill in the art, a functionally equivalent proteins can be produced by recombinant techniques, e.g., by expressing a “functionally equivalent DNA”. As used herein, the term “functionally equivalent DNA” refers to a non-naturally occurring DNA, which encodes a therapeutic agent. For example, many, if not all, of the agents disclosed in Tables 1-3 have known amino acid sequences, which are encoded by naturally occurring nucleic acids. However, due to the degeneracy of the genetic code, more than one nucleic acid can encode the same therapeutic agent. Accordingly, the instant invention embraces therapeutic agents encoded by naturally-occurring DNAs, as well as by non-naturally-occurring DNAs, which encode the same protein as, encoded by the naturally-occurring DNA.

The above-disclosed therapeutic agents and conditions amenable to gene replacement therapy are merely illustrative and are not intended to limit the scope of the instant invention. The selection of a suitable therapeutic agent for treating a known condition is deemed to be within the scope of one of ordinary skill of the art without undue experimentation.

Methods for Introducing Genetic Material into Cells

The exogenous genetic material (e.g., a cDNA encoding one or more therapeutic proteins) is introduced into the cell ex vivo or in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation (Methods in Molecular Biology, Vol. 7, Gene Transfer and Expression Protocols, Ed. E. J. Murray, Humana Press (1991)); DEAE-dextran (supra); electroporation (supra); cationic liposome-mediated transfection (supra); and tungsten particle-faciliated microparticle bombardment (Johnston, S. A., Nature 346:776-777 (1990)). Strontium phosphate DNA co-precipitation (Brash D. E. et al. Molec. Cell. Biol. 7:2031-2034 (1987) is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the -actin promoter (Lai et al., Proc. Natl. Acad. Sci. USA 86: 10006-10010 (1989)), and other constitutive promoters known to those of skill in the art. In addition, many viral promoters function constitutively in eucaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The therapeutic agent can be targeted for delivery to an extracellular, intracellular or membrane location. If it is desirable for the gene product to be secreted from the cells, the expression vector is designed to include an appropriate secretion “signal” sequence for secreting the therapeutic gene product from the cell to the extracellular milieu. If it is desirable for the gene product to be retained within the cell, this secretion signal sequence is omitted. In a similar manner, the expression vector can be constructed to include “retention” signal sequences for anchoring the therapeutic agent within the cell plasma membrane. For example, all membrane proteins have hydrophobic transmembrane regions, which stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

The following discussion is directed to various utilities of the instant invention. For example, the instant invention has utility as an expression system suitable for detoxifying intra- and/or extracellular toxins in situ. By attaching or omitting the appropriate signal sequence to a gene encoding a therapeutic agent capable of detoxifying a toxin, the therapeutic agent can be targeted for delivery to the extracellular milieu, to the cell plasma membrane or to an intracellular location. In one embodiment, the exogenous genetic material containing a gene encoding an intracellular detoxifying therapeutic agent, further includes sequences encoding surface receptors for facilitating transport of extracellular toxins into the cell where they can be detoxified intracellularly by the therapeutic agent. Alternatively, the cells can be genetically modified to express the detoxifying therapeutic agent anchored within the cell plasma membrane such that the active portion extends into the extracellular milieu. The active portion of the membrane-bound therapeutic agent detoxifies toxins, which are present in the extracellular milieu.

In addition to the above-described therapeutic agents, some of which are targeted for intracellular retention, the instant invention also embraces agents intended for delivery to the extracellular milieu and/or agents intended to be anchored in the cell plasma membrane.

The selection and optimization of a particular expression vector for expressing a specific gene product in an isolated cell is accomplished by obtaining the gene, potentially with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

In one embodiment, vectors for cell gene therapy are viruses, such as replication-deficient viruses (described in detail below). Exemplary viral vectors are derived from: Harvey Sarcoma virus; ROUS Sarcoma virus, (MPSV); Moloney murine leukemia virus and DNA viruses (e.g., adenovirus) (Ternin, H., “Retrovirus vectors for gene transfer”, in Gene Transfer, Kucherlapati R, Ed., pp 149-187, Plenum, (1986)).

Replication-deficient retroviruses, including the recombinant lentivirus vectors, are neither capable of directing synthesis of virion proteins or making infectious particles. Accordingly, these genetically altered retroviral expression vectors have general utility for high-efficiency transduction of genes in cultured cells, and specific utility for use in the method of the present invention. The lentiviruses, with their ability to transduce nondividing cells, have general utility for transduction of hepatocytes, cells in cerebrum, cerebellum and spinal cord, and also muscle and other slowly or non-dividing cells. Such retroviruses further have utility for the efficient transduction of genes into cells in vivo. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in Kriegler, M. Gene Transfer and Expression, A Laboratory Manual, W. H. Freeman Co, New York, (1990) and Murray, E. J., ed. Methods in Molecular Biology., Vol. 7, Humana Press Inc., Clifton, N.J., (1991).

The major advantage of using retroviruses, including lentiviruses, for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types (see e.g., Hilberg et al., Proc. Natl. Acad. Sci. USA 84:5232-5236 (1987); Holland et al., Proc. Natl. Acad. Sci. USA 84:8662-8666 (1987); Valerio et al., Gene 84:419-427 (1989). The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome (Miller, D. G., et al., Mol. Cell. Biol. 10:4239-4242 (1990)). While proliferation of the target cell is readily achieved in vitro, proliferation of many potential target cells in vivo is very low.

Yet another viral candidate useful as an expression vector for transformation of cells is the adenovirus, a double-stranded DNA virus. The adenovirus is frequently responsible for respiratory tract infections in humans and thus appears to have an avidity for the epithelium of the respiratory tract (Straus, S., The Adenovirus, H. S. Ginsberg, Editor, Plenum Press, New York, P. 451-496 (1984)). Moreover, the adenovirus is infective in a wide range of cell types, including, for example, muscle and endothelial cells (Larrick, J. W. and Burck, K. L., Gene Therapy. Application of Molecular Biology, Elsevier Science Publishing Co., Inc., New York, p. 71-104 (1991)). The adenovirus also has been used as an expression vector in muscle cells in vivo (Quantin, B., et al., Proc. Natl. Acad. Sci. USA 89:2581-2584 (1992)).

Like the retrovirus, the adenovirus genome is adaptable for use as an expression vector for gene therapy, i.e., by removing the genetic information that controls production of the virus itself (Rosenfeld, M. A., et al., Science 252:431434 (1991)). Because the adenovirus functions in an extrachromosomal fashion, the recombinant adenovirus does not have the theoretical problem of insertional mutagenesis.

Finally, a third virus family adaptable for an expression vector for gene therapy are the recombinant adeno-associated viruses, specifically those based on AAV2, AAV4 and AAV5 (Davidson et al, PNAS, 2000)

Thus, as will be apparent to one of ordinary skill in the art, a variety of suitable viral expression vectors are available for transferring exogenous genetic material into cells. The selection of an appropriate expression vector to express a therapeutic agent for a particular condition amenable to gene replacement therapy and the optimization of the conditions for insertion of the selected expression vector into the cell, are within the scope of one of ordinary skill in the art without the need for undue experimentation.

In an alternative embodiment, the expression vector is in the form of a plasmid, which is transferred into the target cells by one of a variety of methods: physical (e.g., microinjection (Capecchi, M. R., Cell 22:479-488 (1980)), electroporation (Andreason, G. L. and Evans, G. A. Biotechniques 6:650-660 (1988), scrape loading, microparticle bombardment (Johnston, S. A., Nature 346:776-777 (1990)) or by cellular uptake as a chemical complex (e.g., calcium or strontium co-precipitation, complexation with lipid, complexation with ligand) (Methods in Molecular Biology, Vol. 7, Gene Transfer and Expression Protocols, Ed. E. J. Murray, Humana Press (1991)). Several commercial products are available for cationic liposome complexation including Lipofectin™ (Gibco-BRL, Gaithersburg, Md.) (Felgner, P. L., et al., Proc. Natl. Acad. Sci. 84:7413-7417 (1987)) and Transfectam™ (ProMega, Madison, Wis.) (Behr, J. P., et al., Proc. Natl. Acad. Sci. USA 86:6982-6986 (1989); Loeffler, J. P., et al., J. Neurochem. 54:1812-1815 (1990)). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

The instant invention also provides various methods for making and using the above-described genetically-modified cells. In particular, the invention provides a method for genetically modifying cell(s) of a mammalian recipient ex vivo and administering the genetically modified cells to the mammalian recipient. In one embodiment for ex vivo gene therapy, the cells are autologous cells, i.e., cells isolated from the mammalian recipient. As used herein, the term “isolated” means a cell or a plurality of cells that have been removed from their naturally-occurring in vivo location. Methods for removing cells from a patient, as well as methods for maintaining the isolated cells in culture are known to those of ordinary skill in the art.

The instant invention also provides methods for genetically modifying cells of a mammalian recipient in vivo. According to one embodiment, the method comprises introducing an expression vector for expressing a heterologous gene product into cells of the mammalian recipient in situ by, for example, injecting the vector into the recipient.

In one embodiment, the preparation of genetically modified cells contains an amount of cells sufficient to deliver a therapeutically effective dose of the therapeutic agent to the recipient in situ. The determination of a therapeutically effective dose of a specific therapeutic agent for a known condition is within the scope of one of ordinary skill in the art without the need for undue experimentation. Thus, in determining the effective dose, one of ordinary skill would consider the condition of the patient, the severity of the condition, as well as the results of clinical studies of the specific therapeutic agent being administered.

If the genetically modified cells are not already present in a pharmaceutically acceptable carrier they are placed in such a carrier prior to administration to the recipient. Such pharmaceutically acceptable carriers include, for example, isotonic saline and other buffers as appropriate to the patient and therapy.

The genetically modified cells are administered by, for example, intraperitoneal injecting or implanting the cells or a graft or capsule containing the cells in a target cell-compatible site of the recipient. As used herein, “target cell-compatible site” refers to a structure, cavity or fluid of the recipient into which the genetically modified cell(s), cell graft, or encapsulated cell expression system can be implanted, without triggering adverse physiological consequences More than one recombinant gene can be introduced into each genetically modified cell on the same or different vectors, thereby allowing the expression of multiple therapeutic agents by a single cell.

The instant invention further embraces a cell graft. The graft comprises a plurality of the above-described genetically modified cells attached to a support that is suitable for implantation into a mammalian recipient. The support can be formed of a natural or synthetic material.

According to another aspect of the invention, an encapsulated cell expression system is provided. The encapsulated system includes a capsule suitable for implantation into a mammalian recipient and a plurality of the above-described genetically modified cells contained therein. The capsule can be formed of a synthetic or naturally-occurring material. The formulation of such capsules is known to one of ordinary skill in the art. In contrast to the cells which are directly implanted into the mammalian recipient (i.e., implanted in a manner such that the genetically modified cells are in direct physical contact with the cell-compatible site), the encapsulated cells remain isolated (i.e., not in direct physical contact with the site) following implantation. Thus, the encapsulated system is not limited to a capsule including genetically-modified non-immortalized cells, but may contain genetically modified immortalized cells.

The following provides examples of how the Tat-PTD alters the properties of a representative lysosomal protein, β-glucuronidase. Similar results would be expected for all soluble lysosomal proteins. Moreover, the data would also hold for other non-lysosomal proteins that or normally secreted, or to proteins modified to contain a signal sequence to allow for their secretion. The underlying theme is that the inclusion of a PTD onto those sequences will allow for altered and improved biodistribution for therapeutic purposes.

Therefore, the following examples are intended to illustrate but not limit the invention.

### EXAMPLES

### Example 1

### Production of Recombinant vectors

Primer 1 (5′-AAACTCGAGATGGCCCGGGGGTCGGCGGTTGCC-3′) (SEQ ID NO: 1) and primer 2 (5′-TGCTCTAGATCATCTTCGTCGCTGTCTCCGCTTCTTCCTGCCATAACCGCC ACCG-CCAGTAAACGGGCTGTT T TCCAAACA-3′) (SEQ ID NO:2) were used to create the β-glucuronidase-Tat₄₇₋₅₇ fusion protein. Primer 1 and primer 3 (5′TGCTCTAGATCAATAGCCCCTCTTC TTCCGTCT CTGTCGTCGTCTACCGCCACCGCCAGTAAACGGGCTGTTTTCCA AACA-3′) (SEQ ID NO:3) were used to make the β-glucuronidase-Tat₅₇₋₄₇ fusion protein. PCR fragments were digested with XhoI and XbaI and the fragments cloned into similarly cut E1 shuttle plasmids (pPacRSVKpnA; described in (Anderson, et al., (2000) Gene Ther. 7(12):1034-1038)). The resultant plasmids were named pPacRSVβGluc-Tat PTD₄₇₋₅₇ or pPacRSV βGluc-Tat PTD₅₇₋₄₇. Adenoviruses with β-glucuronidase, β-glucuronidase-Tat PTD₄₇₋₅₇ or β-glucuroniase-Tat PTD₅₇₋₄₇ in E1 and eGFP in E3 were produced by co-transfecting PacI linearized pPacRSVβGluc-Tat PTD₄₇₋₅₇, pPacRSVβGluc-Tat PTD₅₇₋₄₇ or pPacRSVβgluc with PacI digested E3 modified Ad5 backbones containing a RSVGFP expression cassette in E3. For ease of discussion the recombinant viruses, Ad5βgluc-Tat₄₇₋₅₇/E3GFP, Ad5βgluc-Tat₅₇₋₄₇/E3GFP or Ad5βgluc/E3GFP are listed as Adβgluc-Tat₄₇₋₅₇, Adβgluc-Tat₅₇₋₄₇ or Adβgluc. Viruses were purified by CsCl gradient ultracentrifugation. Infectious units were determined by plaque assay and particle titers by OD₂₆₀.

### Example 2

### In vitro Studies

HeLa cells were infected with Adβgluc-Tat₄₇₋₅₇, Adβgluc-Tat₅₇₋₄₇ or control Adβgluc at 20 infectious units (i.u.)/cell and supernatants harvested 72 h later. β-Glucuronidase activity was quantified using the previously described fluorometric assay. Briefly, aliquots were reacted in 10 mM 4-methylumbellifryl-β-D-glucuronidase (Sigma, St. Louis, Mo.) in 0.1 M sodium acetate (pH 4.8) for 1 h at 37° C. Reactions were stopped by addition of 2 ml of 320 mM glycine in 200 mM carbonate buffer, pH 10.0 (Glaser, et al., (1973) J.Lab.Clin.Med. 82:969-977).

Fluorescence was measured at 415 mn after excitation at 360 nm (TD-700 Fluorometer; Turner Design, Sunnyvale, Calif.). β-Glucuronidase activity is expressed as nanomoles of 4-methylumbellferone released per hour (FLU) per mg protein. Purified β-glucuronidase (kindly provided by William Sly, Washington University, St. Louis Mo.) was used as standard. Protein concentrations were determined using the Bio-Rad DC protein assay (Bio-Rad Laboratories, Hercules, Calif.).

NIH 3T3 or A549 cells (500,000 cells plated the day before) were incubated with 5500 units of β-glucuronidase-Tat₄₇₋₅₇, β-glucuronidase-Tat₅₇₋₄₇ or β-glucuronidase in the presence or absence of D-mannose-6-phosphate (10 mM) for 2 h at 37 or 4° C. After incubation cells were harvested and lysates prepared for fluorometric enzyme assay, or stained for β-glucuronidase activity in situ. For β-glucuronidase staining, cells were washed in PBS, fixed in 2% paraformaldehyde for 15 min, washed twice in PBS, twice with 0.05M sodium acetate, pH 4.5, for 5 min, and then incubated in 0.25 mM Napth-As-Bi-β-glucuronide (Sigma) in the same buffer for 40 min. Cells or tissues (below) were then stained for 30 min at 37° C. with 0.25 mM Napth-As-Bi-β-glucuronide in 0.05 M sodium acetate, pH 5.2, with 1/500 2% hexazotized pararosaniline (Sigma).

### Example 3

### In vivo Studies

β-Glucuronidase-deficient mice were obtained from the Jackson Laboratory 30 (Bar Harbor, Me.) and from our own breeding colony. The genotype for the latter was confirmed by morphological and genetic analyses. The animals were between 8 and 10 weeks old and weighed 16-24 g. C57BL/6 wild-type mice were purchased from Harlan Sprague (Indianapolis, Ind.).

Adβgluc-Tat₄₇₋₅₇, Adβgluc-Tat₅₇₋₄₇ or Adβgluc were injected into the tail vein (2×10⁹ i.u.) of β-glucuronidase deficient mice. Adβgluc-Tat₄₇₋₅₇, Adβgluc-Tat₅₇₋₄₇ or Adβgluc (2×10⁷ i.u. total) were injected into the right striatum or right lateral ventricle of C57BL/6 mice or β-glucuronidase deficient mice as described earlier (Stein, et al., (1999) J. Virol. 73(4):3424-3429). Animals were sacrificed 10 days after intravenous (n=3/group), striatal (n=5/group) or ventricular injection (n=5/group). Tissues were sonicated, placed in lysis solution (Sands, et al., (1994) J.Clin.Invest. 93:2324-2331) and centrifuged at 12000× g for 20 min. Aliquots were assayed using the fluorometric assay described above. For in situ enzyme assays, tissues were harvested, sectioned, and stained in situ for β-glucurondase activity as described above.

Coronal brain sections were photographed with Adobe Photoshop (Adobe system, Mountain View, Calif.), and the photos imported into NIH Image. Color thresh-holding was used, and the percentage of brain positive for activity calculated by dividing the area of staining by the total area (adjusted for ventricular size). In all cases a minimum of 2 mm (rostral to caudal) of cerebrum surrounding the injection site was scanned.

All publications, patents and patent applications are incorporated herein by reference. While in the foregoing specification this invention has been described in relation to certain embodiments, and many details have been set forth for purposes of illustration, it will be apparent to those skilled in the art that the invention is susceptible to additional embodiments and that certain of the details described herein may be varied considerably without departing from the basic principles of the invention.

### REFERENCES

1. Meikle, P. J., Hopwood, J. J., Clague, A. E. and Carey, W. F. 1999. Prevalence of lysosomal storage disorders. JAMA 281(3):249-254.

2. O'Connor, L. H., Erway, L. C., Vogler, C. A., Sly, W. S., Nicholes, A., Grubb, J., Holmberg, S. W., Levy, B. and Sands, M.S. 1998. Enzyme replacement therapy for murine mucopolysaccharidosis Type VII leads to improvements in behavior and auditory function. J.Clin.Invest. 101:1394-1400.

3. Frisella, W. A., O'Connor, L. H., Vogler, C. A., Roberts, M., Walkley, S., Levy, B., Daly, T. M. and Sands, M. S. 2001. Intracranial injection of recombinant adeno-associated virus improves cognitive function in a murine model of mucopolysaccharidosis type VII. Mol. Ther. (In Press)

4. Fawell, S., Seery, J., Daikh, Y., Moore, C., Chen, L. L., Pepinsky, B. and Barsoum, J. 1994. Tat-mediated delivery of heterologous proteins into cells. Proc.Natl.Acad.Sci.U.S.A. 91:664-668.

5. Schwarze, S. R., Ho, A., Vocero-Akbani, A. and Dowdy, S. F. 1999. In vivo protein transduction: delivery of a biologically active protein into the mouse. Science 285:1569-1572.

6. Derossi, D., Calvet, S., Trembleau, A., Brunissen, A., Chassaing, G. and Prochiantz, A. 1996. Cell internalization of the third helix of the antennapedia homeodomain is receptor-independent. J.Biol. Chem. 271(30):18188-18193.

7. Ghodsi, A., Stein, C., Derksen, T., Yang, G., Anderson, R. D. and Davidson, B. L. 1998. Extensive β-glucuronidase activity in murine CNS after adenovirus mediated gene transfer to brain. Hum. Gene Ther. 9:2331-2340.

8. Mann, D. A. and Frankel, A. D. 1991. Endocytosis and targeting of exogenous HIV-1 tat protein. EMBO J 10(7):1733-1739.

9. Stein, C. S., Ghodsi, A., Derksen, T. and Davidson, B. L. 1999. Systemic and central nervous system correction of lysosomal storage in mucopolysaccharidosis type VII mice . J. Virol. 73(4):3424-3429.

10. Ghodsi, A., Stein, C., Derksen, T., Martins, I., Anderson, R. D. and Davidson, B. L. 1999. Systemic hyperosmolality improves β-glucuronidase distribution and pathology in murine MPS VII brain following intraventricular gene transfer. Exp.Neurol. 160:109-116.

11. Davidson, B. L., Stein, C. S., Heth, J. A., Martins, I., Kotin, R. M., Derksen, T. A., Zabner, J., Ghodsi, A. and Chiorini, J. A. 2000. Recombinant AAV type 2, 4 and 5 vectors: transduction of varient cell types and regions in the mammalian CNS. Proc.Natl.Acad.Sci.U.S.A. 97(7):3428-3432.

12. Anderson, R. D., Haskell, R. E., Xia, H., Roessler, B. J. and Davidson, B. L. 2000. A simple method for the rapid generation of recombinant adenovirus vectors. Gene Ther. 7(12):1034-1038.

13. Glaser, J. H. and Sly, W. S. 1973. Beta-glucuronidase deficiency mucopolysaccharidosis: Methods for enzymatic diagnosis. J.Lab.Clin.Med. 82:969-977.

14. Sands, M. S., Vogler, C., Kyle, J. W., Grubb, J. H., Levy, B., Galvin, N., Sly, W. S. and Birkenmeier, E. H. 1994. Enzyme replacement therapy for murine mucopolysaccharidosis type VII. J.Clin.Invest. 93:2324-2331.

1-2. (canceled)

3. The method of claim 53, wherein the lysosomal enzyme is a soluble lysosomal enzyme.

4. The method claim 3, wherein the soluble lysosomal enzyme is beta-glucuronidase, pepstatin insensitive protease or palmitoyl protein thioesterase.

5. The method of claim 3, wherein the soluble lysosomal enzyme is beta-glucuronidase.

6-13. (canceled)

14. The method of claim 53, wherein the PTD is Tat PTD.

15. The method of claim 14, wherein the Tat PTD is Tat₄₇₋₅₇.

16-17. (canceled)

18. The method of claim 61, wherein the lysosomal enzyme is a soluble lysosomal enzyme.

19. The method of claim 18, wherein the soluble lysosomal enzyme is beta-glucuronidase, pepstatin insensitive protease or palmitoyl protein thioesterase.

20. The method of claim 19, wherein the soluble lysosomal enzyme is beta-glucuronidase

21-18. (canceled)

29. The method of claim 61, wherein the PTD is Tat PTD.

30. The method of claim 29, wherein the Tat PTD is Tat₄₇₋₅₇.

31. The method of claim 61, wherein the vector is an adenoviral vector.

32. The method of claim 61, wherein the vector is an adeno-associated virus vector.

33. The method of claim 61, wherein the vector is a recombinant lentivurs or retrovirus vector.

34-49. (canceled)

50. The method of claim 61, wherein the cell is human.

51. The method of claim 61, wherein the cell is from spleen, kidney, lung, heart, liver or brain.

52. The method of claim 61, wherein the cell is a stem or progenitor cell.

53. A method of treating a lysosomal storage disease (LSD) in a mammal comprising administering a polynucleotide comprising a nucleic acid sequence encoding a lysosomal enzyme operably linked to a nucleic acid sequence encoding a protein transduction domain (PTD).

54. The method of claim 53, wherein the mammal is human.

55. (canceled)

56. The method of claim 53, wherein the LSD is infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.

57-58. (canceled)

59. A method of treating a lysosomal storage disease in a mammal comprising administering an expression vector comprising a nucleic acid sequence encoding a lysosomal enzyme operably linked to a nucleic acid sequence encoding a PTD.

60. (canceled)

61. A method of treating a lysosomal storage disease in a mammal comprising administering a mammalian cell comprising an expression vector comprising a nucleic acid sequence encoding a lysosomal enzyme operably linked to a nucleic acid sequence encoding a PTD.

62. The method of claim 59, wherein the lysosomal enzyme is a soluble lysosomal enzyme.

63. The method of claim 62, wherein the soluble lysosomal enzyme is beta-glucuronidase, pepstatin insensitive protease or palmitoyl protein thioesterase.

64. The method of claim 63, wherein the soluble lysosomal enzyme is beta-glucuronidase

65. The method of claim 59, wherein the PTD is Tat PTD.

66. The method of claim 65, wherein the Tat PTD is Tat₄₇₋₅₇.

67. The method of claim 59, wherein the vector is an adenoviral vector.

68. The method of claim 59, wherein the vector is an adeno-associated virus vector.

69. The method of claim 59, wherein the vector is a recombinant lentivurs or retrovirus vector.

70. The method of claim 59, wherein the mammal is human.

71. The method of claim 59, wherein the LSD is infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.

72. The method of claim 61, wherein the mammal is human.

73. The method of claim 61, wherein the LSD is infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.


SEQUENCE LISTING (3 sequences)

SEQ ID NO: 1 - DNA, 33 bp, Adenovirus
aaactcgagatggcccgggggtcggcggttgcc

SEQ ID NO: 2 - DNA, 81 bp, Adenovirus
tgctctagatcatcttcgtcgctgtctccgcttcttcctgccataaccgccaccgccagtaaacgggctgttttccaaaca

SEQ ID NO: 3 - DNA, 81 bp, Adenovirus
tgctctagatcaatagcccctcttcttccgtctctgtcgtcgtctaccgccaccgccagtaaacgggctgttttccaaaca